(19) United States
(12) Patent Application Publication
(10) Pub. No.: US 2026/0363564 A1
(43) Pub. Date: Nov. 19, 2026

(54) Heart Rate Estimation Device and Headset System

(71) Applicant: FOSTER ELECTRIC COMPANY, LIMITED, Tokyo (JP)

(72) Inventors: Yo Hibino, Akishima-shi, Tokyo (JP); Tomoaki Kawabe, Akishima-shi, Tokyo (JP); Hidehito Miho, Akishima-shi, Tokyo (JP)

(21) Appl. No.: 19/124,079

(22) PCT Filed: Oct. 4, 2023
(86) PCT No.: PCT/JP2023/036285

§ 371 Date: Apr. 24, 2025

(30) Foreign Application Priority Data
Oct. 26, 2022 (JP) 2022-171796

(51) Int. Cl. A61B 5/024; A61B 5/00

(52) U.S. Cl. CPC A61B 5/024; A61B 5/6817; A61B 5/7257; A61B 2562/0204

(57) ABSTRACT

A heart rate estimation device includes a feature point extraction unit that extracts a waveform feature point from a waveform expressing pressure fluctuation in a sealed space; a pulse waveform generation unit that generates a pulse waveform forming a pulse at a same timing as the waveform feature point; a frequency analysis unit that executes frequency analysis of the pulse waveform; and a heart rate estimation unit that estimates a heart rate by analyzing a frequency feature point obtained from a result of the frequency analysis.

### TECHNICAL FIELD

The technique of the present disclosure relates to a heart rate estimation device and a headset system.

### BACKGROUND ART

Attempts to collect biological information from the head of a human body using an earphone are actively underway. Among biological information, it is particularly desired to collect information with high accuracy with respect to head movement, core body temperature, and heartbeat.

In processing for estimating a heart rate, it is important to eliminate erroneous detection due to noise generated by body movement. Thus, in an optical heart rate estimation method, a method is known in which plural light sources and sensors are used to observe the same spot from plural directions, and noise due to body movement is removed (see, for example, U.S. Pat. No. 8,998,815).

### SUMMARY OF THE INVENTION

### Problem to be Solved by the Invention

However, in a case in which the heart rate is estimated from pressure fluctuations in a sealed space, since pressure fluctuations in the same space are substantially uniform, an effect for noise reduction cannot be expected, even if plural pressure sensors or microphones are used.

As a result, it is necessary to estimate the heart rate by analyzing pressure fluctuations sensed by a pressure sensor or a microphone in a state including noise. However, in pressure fluctuations in a sealed space, fluctuations due to body movement are often significantly greater than inflation and contraction of blood vessels due to blood flow.

Thus, even when general frequency analysis (spectrum analysis) is performed on pressure fluctuations in a sealed space, characteristics attributable to body movement, artifacts, and the like become dominant, and the problem arises that cyclic feature points caused by inflation and contraction of a blood vessel are buried.

An object of the technique of the present disclosure is to provide a heart rate estimation device and a headset system that are capable of accurately estimating a heart rate from pressure fluctuations in a sealed space.

### Means for Solving the Problem

An aspect of the present disclosure is a heart rate estimation device configured including: a feature point extraction unit that extracts a waveform feature point from a waveform expressing pressure fluctuation in a sealed space; a pulse waveform generation unit that generates a pulse waveform forming a pulse at a same timing as the waveform feature point; a frequency analysis unit that executes frequency analysis of the pulse waveform; and a heart rate estimation unit that estimates a heart rate by analyzing a cyclic feature point obtained from a result of the frequency analysis.

An aspect of the present disclosure is a headset system configured including: the above-described heart rate estimation device; and a headset including: a hollow housing to be worn at an ear of a user; a driver for acoustic signal output provided at an interior of the housing; an optical detection unit provided at the interior of the housing and rearward of the driver; and an output unit that outputs, to the heart rate estimation device, as a waveform expressing pressure fluctuation in a sealed space, a signal output from the optical detection unit.

### Effect of Invention

As explained above, according to the heart rate estimation device and the headset system of the technique of the present disclosure, the heart rate can be accurately estimated from pressure fluctuations in a sealed space.

### BRIEF DESCRIPTION OF THE DRAWINGS

FIG. 1A is a diagram illustrating an example of a waveform of a signal output from a microphone.

FIG. 1B is a diagram illustrating an example of waveform feature points extracted from a waveform of a signal output from a microphone.

FIG. 1C is a diagram illustrating an example of identical timing to waveform feature points.

FIG. 1D is a diagram illustrating a method of generating a pulse waveform.

FIG. 1E is a diagram illustrating an example of a pulse waveform.

FIG. 1F is a diagram illustrating an example of a result of frequency analysis on a pulse waveform.

FIG. 1G is a diagram illustrating an example of cyclic feature points obtained from a result of frequency analysis.

FIG. 2A is a diagram illustrating an example of waveform feature points extracted from a waveform of pressure fluctuation in a sealed space at a time of rest.

FIG. 2B is a diagram illustrating an example of cyclic feature points obtained from a result of frequency analysis.

FIG. 3A is a diagram illustrating an example of waveform feature points extracted from a waveform of pressure fluctuation in a sealed space at a time of body movement.

FIG. 3B is a diagram illustrating an example of cyclic feature points obtained from a result of frequency analysis.

FIG. 4A is a diagram illustrating an example of a result of frequency analysis by a discrete Fourier transform.

FIG. 4B is a diagram illustrating an example of a result of frequency analysis by a fast Fourier transform.

FIG. 5 is a cross-section illustrating an overall configuration of a headset according to a first exemplary embodiment of the technique of the present disclosure.

FIG. 6 is a block diagram illustrating a configuration of an information processing unit according to a first exemplary embodiment of the technique of the present disclosure.

FIG. 7 is a diagram illustrating a method of extracting a zero crossing point as a waveform feature point.

FIG. 8 is a diagram illustrating an example of provisional peaks and provisional troughs detected from a waveform of a signal.

FIG. 9 is a diagram illustrating an example of provisional peaks and provisional troughs after removal of provisional peaks and provisional troughs that are presumed to have been erroneously detected.

FIG. 10 is a flowchart illustrating the content of heart rate estimation processing by an information processing unit according to a first exemplary embodiment of the technique of the present disclosure.

FIG. 11 is a cross-section illustrating an overall configuration of a headset according to a second exemplary embodiment of the technique of the present disclosure.

FIG. 12 is a cross-section illustrating an overall configuration of a headset according to an example.

### DESCRIPTION OF THE INVENTION

Detailed explanation follows regarding exemplary embodiments of the technique of the present disclosure, with reference to the drawings.

### First Exemplary Embodiment

### Overview of First Exemplary Embodiment of Technique of Present Disclosure

In processing for estimating the heart rate, it is important to eliminate erroneous detection of the heartbeat due to noise generated by body motion, an artifact, or the like.

In the present exemplary embodiment, frequency analysis is not directly performed on a waveform of pressure fluctuation in a sealed space, and waveform feature points, which are characteristic points of the waveform, are extracted, and a pulse waveform in which pulses are formed at the same timing as the waveform feature points is generated. Frequency analysis is performed on the pulse waveform, and the heart rate is estimated by analyzing cyclic feature points, which are characteristic points of the frequency analysis.

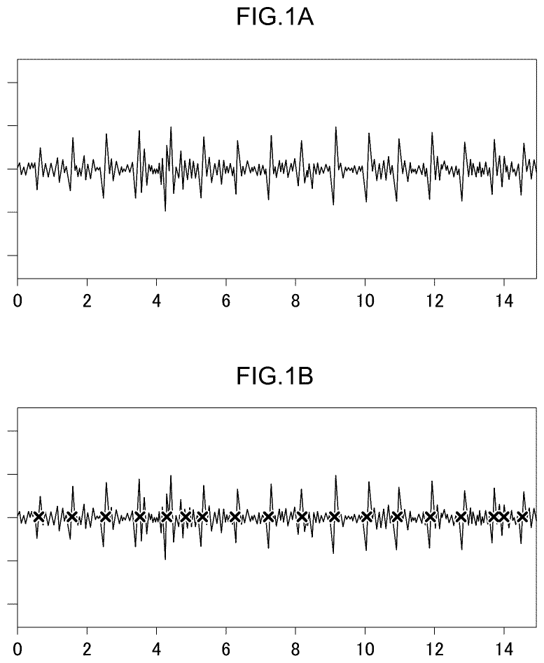

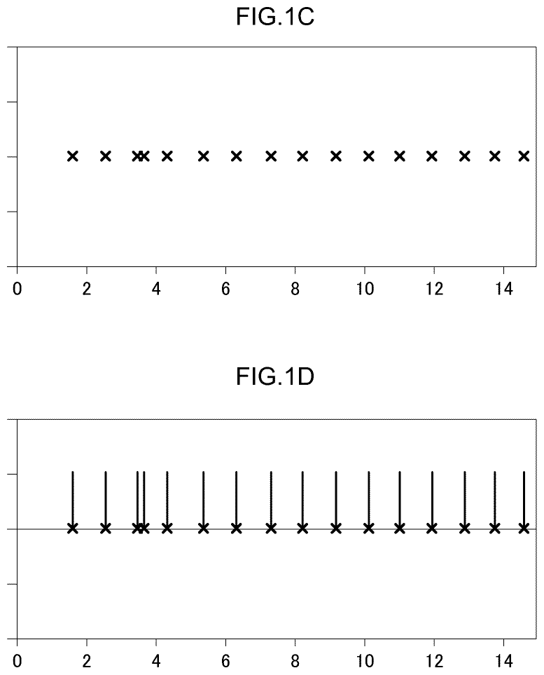

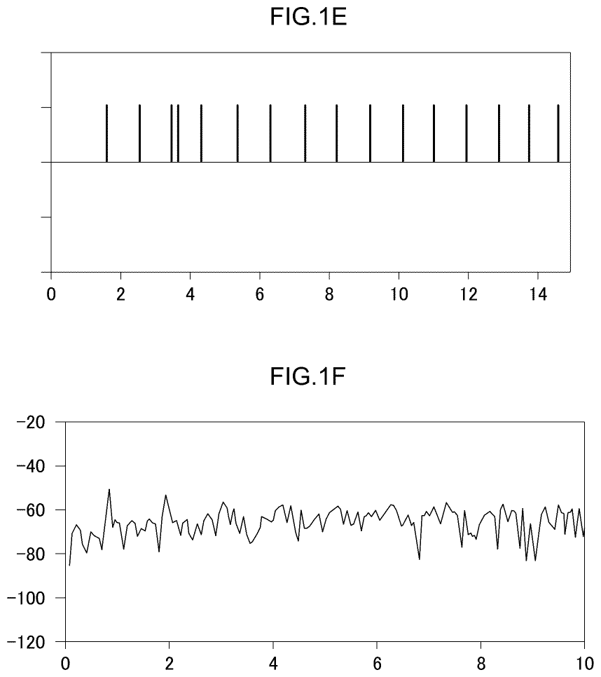

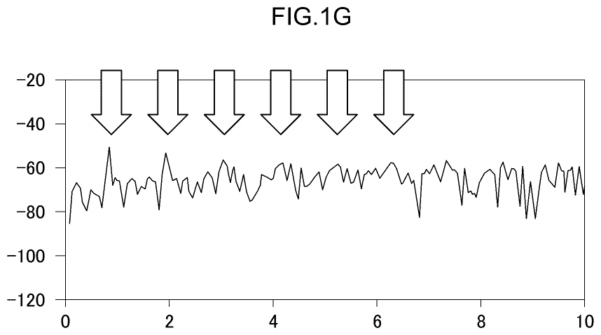

Specifically, a waveform of pressure fluctuation in the sealed space such as illustrated in FIG. 1A is acquired, and waveform feature points are extracted as illustrated in FIG. 1B (see the x-marks in FIG. 1B). As illustrated in FIGS. 1C and 1D, a pulse waveform is generated in which pulses are formed at the same timing as the waveform feature points. Frequency analysis is performed on a pulse waveform such as that illustrated in FIG. 1E, and a frequency analysis result illustrated in FIG. 1F is obtained. The heart rate is estimated from the cyclic feature points indicated by the arrow marks in FIG. 1G.

A waveform feature point caused by a large pressure change in the sealed space due to body movement or the like, and a waveform feature point caused by a small pressure change in the sealed space due to inflation and contraction of a blood vessel are converted to the same fluctuation level, and frequency analysis is performed. Thus, even in cases in which, with conventional methods, they would be buried in the frequency characteristics of body movement, it is easy to distinguish cyclic feature points that have occurred owing to inflation and contraction of a blood vessel, enabling heart rate to be accurately estimated, even in states in which body movement occurs.

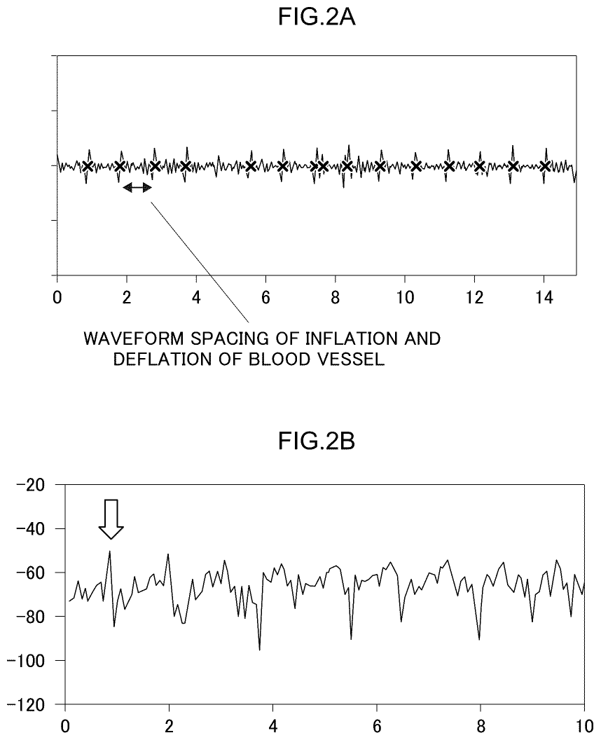

Here, FIG. 2A shows an example of waveform feature points extracted from a waveform of pressure fluctuation in a sealed space at a time of rest. In this example, the pulse rate is estimated to be 64 ppm (1.07 Hz) from the waveform spacing of inflation and deflation of the blood vessel. At this time, in the present exemplary embodiment, a result of frequency analysis such as illustrated in FIG. 2B is obtained, and a peak (see the arrow mark in FIG. 2B) clearly appears in a band calculated from the waveform spacing, which is extracted as a cyclic feature point. It is understood that this cyclic feature point corresponds to 64 ppm (1.07 Hz).

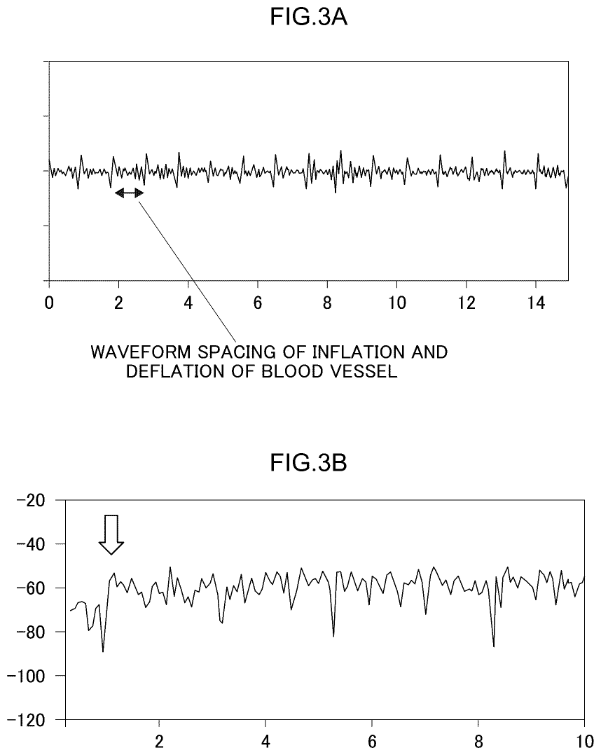

Further, FIG. 3A shows an example of waveform feature points extracted from a waveform of pressure fluctuation in a sealed space at a time of body movement. In this example, the pulse rate is estimated to be 68 ppm (1.13 Hz) from the waveform spacing of inflation and deflation of the blood vessel. At this time, in the present exemplary embodiment, a result of frequency analysis such as illustrated in FIG. 3B is obtained, and a peak (see the arrow mark in FIG. 3B) clearly appears in a band calculated from the waveform spacing, which is extracted as a cyclic feature point. It is understood that this cyclic feature point corresponds to 68 ppm (1.13 Hz).

Further, the amplitude of the pulse waveform may be changed using an arbitrary value, or may be fixed to a fixed value. Fast Fourier transform may be used for frequency analysis, but discrete Fourier transform (DFT) is preferably used.

For the purpose of estimating heart rate, since it is sufficient to be able to extract a frequency with a high intensity, the discrete Fourier transform for obtaining the frequency characteristic Ff of the pulse wave X(n) can be simplified as follows, omitting operations for normalizing the output, such as square root calculation of the square mean.

$$F ( f ) = {( {\sum }_{n = 0}^{N - 1} X ( n ) \cdot cos ( 2 π \frac{nf}{{f}_{s}} ) )}^{2} + {( {\sum }_{n = 0}^{N - 1} X ( n ) \cdot sin ( 2 π \frac{nf}{{f}_{s}} ) )}^{2} \qquad ( 1 )$$

In Expression (1), N indicates the number of samples of the pulse wave X(n), fₛ indicates the sampling frequency of X(n), and f indicates the frequency being analyzed.

Note that since X(n) is a pulse wave, there is no need to perform arithmetic operations because X(n) is zero except for the pulse point n at which a pulse exists, and further, since, in cases in which the amplitude is a fixed value, the sine value/cos value is obtained only by table reference, intensity can be obtained by performing an N ALU/MUX operation (complex processing of table reference, multiplication, and addition) for each frequency f. In addition thereto, it is possible to perform calculation that is limited to only the frequency f required for heart rate estimation. This enables a significant reduction in computational complexity compared to when a fast Fourier transform is performed (computational complexity: N log₂ N).

An example in a case in which Equation (1) is applied to the above-described pulse waveform of FIG. 1E is shown below.
Sampling frequency fₛ=100 Hz
Sample length N=2048
Number of pulse points: 45 points
Analysis frequency: 0.5≤f≤3.0 (equivalent to 30 ppm to 180 ppm)

The cost required for calculating the frequency characteristic Xf in the pulse waveform of FIG. 1E described above was as follows.

The capacity of the table memory is 2048×16 bits. The number of ALU/MUX operations is 4500 (=45 (number of pulse points)×50 (number of analysis bands)×2 (real number portion+imaginary number portion)). Here, 45 is the number of pulse points, 50 is the number of analysis bands, and 2 is the pair of a real number portion and an imaginary number portion.

The cost required when obtained by fast Fourier transform is as follows.

The capacity of the table memory is 2048×16 bits. The number of ALU/MUX operations is 40960 (=2048×log₂(2048)×2).

In this manner, the advantageous effects of the present exemplary embodiment can be confirmed, as compared to the cost required when a fast Fourier transform is performed.

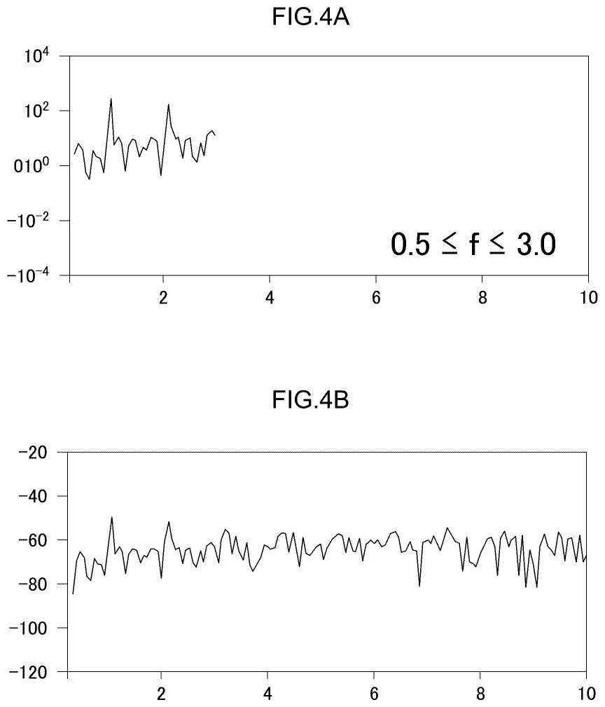

FIGS. 4A and 4B show the obtained frequency characteristics. FIG. 4A is a diagram illustrating an example of a result of frequency analysis by a discrete Fourier transform, illustrating an example in which the analysis frequency f is 0.5 Hz to 3.0 Hz. FIG. 4B is a diagram illustrating an example of a result of frequency analysis by a fast Fourier transform.

While the result of frequency analysis by the discrete Fourier transform is different from the result of frequency analysis by the fast Fourier transform on the Y-axis scale, since the shapes coincide with each other, it can be confirmed that there is no problem even if the shape is simplified by a discrete Fourier transform.

### Configuration of Headset System of First Exemplary Embodiment of Technique of Present Disclosure

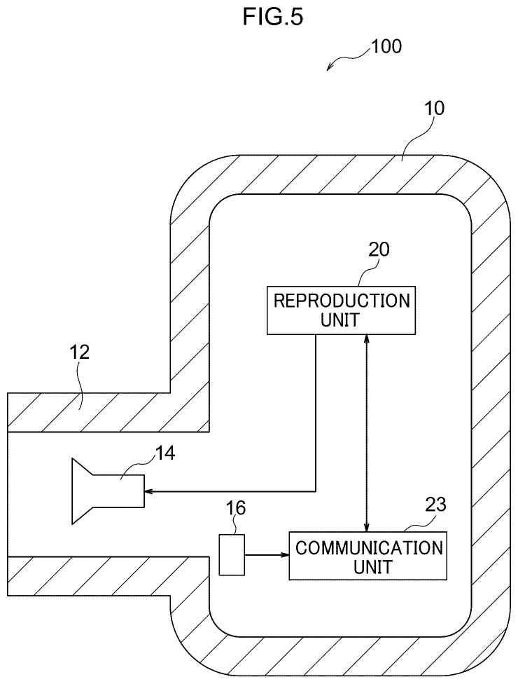

A headset system according to a first exemplary embodiment of the technique of the present disclosure includes a headset 100 illustrated in FIG. 5.

As illustrated in FIG. 5, the headset 100 includes a hollow housing 10 that is worn at the ear of a user, and that houses various functional components internally. The headset 100 includes a tube-shaped external ear canal insertion portion 12 that is a portion of the housing 10, and that includes a hollow portion provided at a portion at the ear canal side of the housing 10 when worn at the car of a user.

Further, the headset 100 includes a driver 14 for outputting sound signals, which is provided inside the housing 10.

Further, the headset 100 is provided with a microphone 16 provided so as to collect signals propagated to the hollow portion of the external ear canal insertion portion 12, a reproduction unit 20 that outputs sound signals from the driver 14, and a communication unit 23 that transmits an output of the microphone 16 to an information processing unit 50.

The reproduction unit 20 and the communication unit 23 are mounted on a main board (not illustrated) disposed inside the housing 10.

When no sound signal is output from the driver 14, the signal output from the microphone 16 is transmitted to the information processing unit 50 by the communication unit 23. Here, when no sound signal is output from the driver 14, the signal output from the microphone 16 is a waveform representing pressure fluctuation in the sealed space formed by the hollow portion of the housing 10 and the external ear canal of the user, and is a waveform illustrated in, for example, FIG. 1A described above.

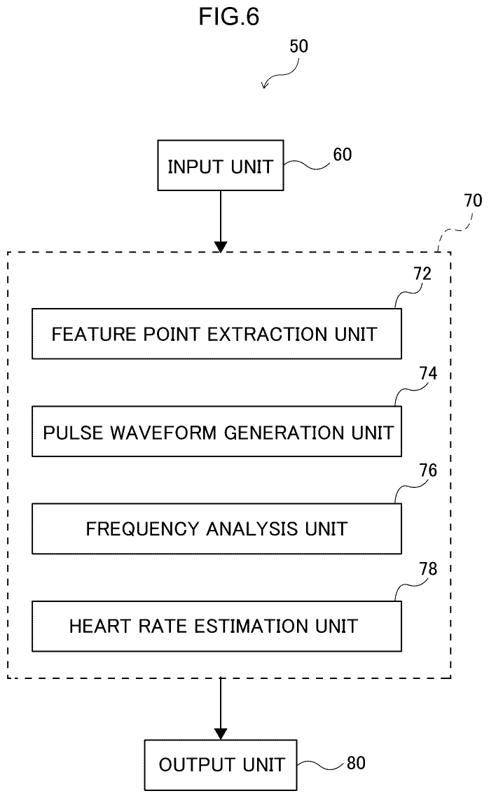

A headset system according to the first exemplary embodiment of the technique of the present disclosure includes the information processing unit 50 illustrated in FIG. 6. Note that the information processing unit 50 is an example of a heart rate estimation device.

The information processing unit 50 is configured by a portable terminal, a computer terminal, or the like, or the information processing unit 50 is incorporated in a headset system. Here, portable terminals include smartphone terminals, cellular phones, and personal digital assistant (PDA) terminals. Computer terminals include laptop and book-type computer terminals, and desktop-type computer terminals. The information processing unit 50 may be provided inside the housing 10 of the headset 100, or may be provided separately from the headset 100.

The information processing unit 50 is configured by a computer including a CPU, a RAM, and a ROM storing a program for executing a heart rate estimation processing routine, which is described below, and is functionally configured as follows.

As illustrated in FIG. 6, the information processing unit 50 includes an input unit 60, a calculation unit 70, and an output unit 80. The calculation unit 70 includes a feature point extraction unit 72, a pulse waveform generation unit 74, a frequency analysis unit 76, and a heart rate estimation unit 78.

The input unit 60 receives input of a signal output from the microphone 16, received from the headset 100.

As illustrated in FIG. 1B above, the feature point extraction unit 72 extracts waveform feature points from a signal output from the microphone 16.

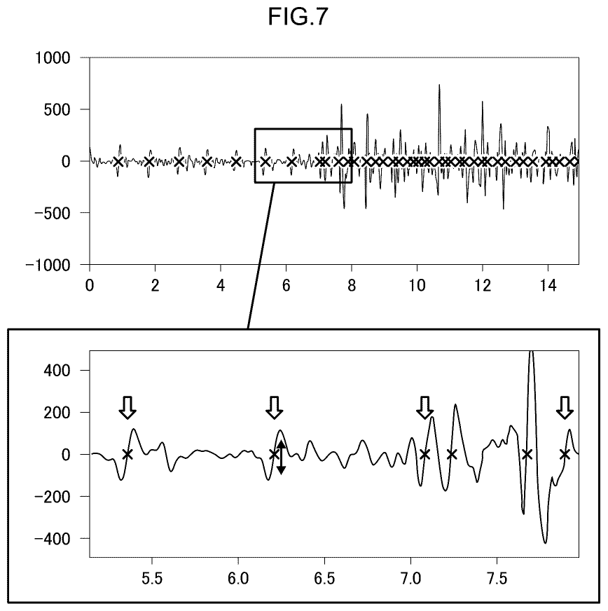

Specifically, the feature point extraction unit 72 extracts, as a waveform feature point, a zero crossing having a fluctuation width equal to or greater than a threshold value, from a signal output from the microphone 16, the waveform representing pressure fluctuation in the sealed space (see the x-marks in FIG. 7).

The lower diagram of FIG. 7 is an enlarged view of the rectangular frame portion in the upper diagram of FIG. 7. In the lower diagram of FIG. 7, of the zero crossing points having a fluctuation width greater than or equal to a threshold value before and after a zero crossing, the zero crossing points indicated by the arrow marks are the zero crossing points estimated to be due to a heartbeat. The other zero crossing points are zero crossing points that are estimated to be due to body movement.

Note that the threshold value of the fluctuation width in the zero crossing point extraction may be varied in accordance with the situation. Note that since the periodicity of all the zero crossing points extracted as a zero crossing with a fluctuation width greater than or equal to the threshold value appears in the frequency characteristic, cyclic feature points appear in the cycle of a heartbeat, even in cases in which a heartbeat and a zero crossing point due to body movement are intermingled. However, the more that waveform feature points due to body movement appear, cyclic feature points due to body movement also appear to the same extent, and therefore, it is more favorable if there are as few waveform feature points due to body movement as possible; however, it is not essential to exclude waveform feature points due to body movement.

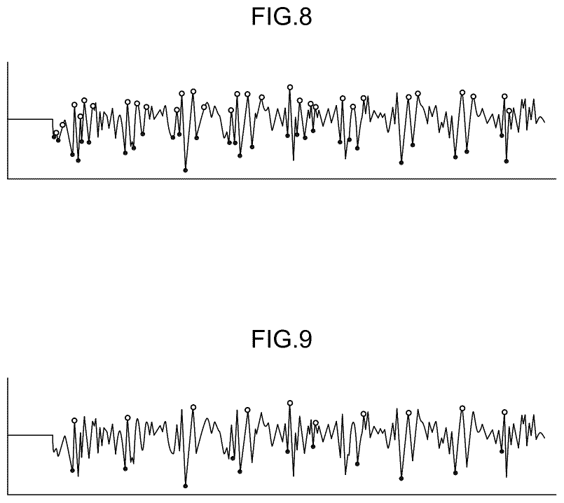

Further, as long as a technique is capable of extracting waveform feature points including points that are linked to a heartbeat, waveform feature points other than a zero crossing may be extracted. For example, peak points and trough points may be extracted as waveform feature points from a waveform representing pressure fluctuation in a sealed space. In such cases, peak points and trough points are detected from a waveform representing pressure fluctuation in a sealed space (FIG. 8), and from among the detected peak points and trough points, peak points and trough points estimated to have been erroneously detected are removed (FIG. 9). More specifically, as illustrated in FIG. 8, points that exceed the running average of the respective upper limits are extracted as provisional peak points (see the white circle portions) from the waveform representing pressure fluctuation in the sealed space. Points that are below the running average of the respective lower limits are extracted as provisional trough points (see the black circle portions). In consideration of the time difference and the amplitude amount from the provisional trough point to the provisional peak point, the spacing between valid provisional peak points, and the spacing between valid provisional trough points, provisional peak points and provisional trough points that are estimated to have been erroneously detected are removed.

Further, a peak point and a trough point may be extracted from a waveform representing pressure fluctuation in a sealed space using machine learning, and used as waveform feature points. For example, peak points and trough points may be extracted using the method described in Reference Document 1.

[Reference Document 1]: “Robust ECG R-peak Detection Using LSTM”, Juho Laitala et al., SAC '20: Proceedings of the 35th Annual ACM Symposium on Applied Computing. March 2020, Pages 1104-1111, https://doi.org/10.1145/3341105.3373945

The pulse waveform generation unit 74 generates a pulse waveform in which a pulse of a predetermined amplitude is formed at the same timing as a waveform feature point. As illustrated in FIG. 1D described above, for example, pulse waveforms are generated in which pulses with a constant amplitude are formed at the same timing as the waveform feature points. Note that the pulse of the pulse waveform does not need to have a constant amplitude, and may be changed using any given value.

The frequency analysis unit 76 performs frequency analysis on the pulse waveform. Specifically, the frequency analysis unit 76 performs frequency analysis by discrete Fourier transform, and obtains frequency characteristics as illustrated in FIG. 1F. Note that instead of a discrete Fourier transform, a fast Fourier transform may be used.

The heart rate estimation unit 78 estimates the heart rate by analyzing cyclic feature points obtained from the results of the frequency analysis.

Specifically, as illustrated in FIG. 1G described above, the heart rate estimation unit 78 extracts, as cyclic feature points, cycles corresponding to frequencies having a frequency component greater than a threshold value, from the results of the frequency analysis. The heart rate estimation unit 78 specifies cyclic feature points corresponding to a heart rate interval, from among the extracted cyclic feature points, and estimates a heart rate per unit time based on the specified cyclic feature points.

### Operation of Headset System of First Exemplary Embodiment of Technique of Present Disclosure

When the housing 10 of the headset 100 is worn at the ear of the user, an instruction to estimate the heart rate is received from the information processing unit 50 of the user by wireless communication. At this time, in the headset 100, the communication unit 23 transmits a signal output from the microphone 16 when no sound signal is output from the driver 14, to the information processing unit 50.

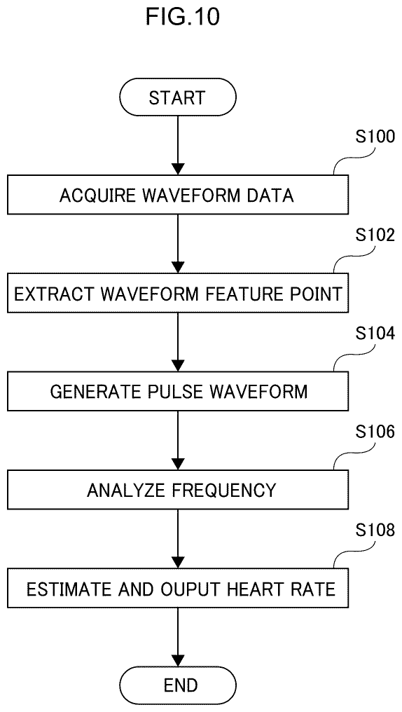

Further, when the information processing unit 50 receives the signal output from the microphone 16, the information processing unit 50 executes heart rate estimation processing as illustrated in FIG. 10.

First, in step S100, the input unit 60 acquires the signal output from the microphone 16 received from the headset 100.

In step S102, the feature point extraction unit 72 extracts waveform feature points from the signal output from the microphone 16.

In step S104, the pulse waveform generation unit 74 generates a pulse waveform in which a pulse of a predetermined amplitude is formed at the same timing as the waveform feature points.

In step S106, the frequency analysis unit 76 performs frequency analysis on the pulse waveform.

In step S108, the heart rate estimation unit 78 estimates the heart rate by analyzing cyclic feature points obtained from the results of the frequency analysis, displays the heart rate at the output unit 80, and ends the heart rate estimation processing.

As explained above, according to the headset system according to the first exemplary embodiment of the present disclosure, the heart rate estimation device extracts waveform feature points from a signal of a waveform representing pressure fluctuation in a sealed space, that is output from a microphone of the headset. The heart rate estimation device estimates the heart rate by generating a pulse waveform in which pulses are formed at the same timing as the waveform feature points, and analyzing cyclic feature points obtained from the results of performing frequency analysis on the pulse waveform. This enables the heart rate to be accurately estimated from pressure fluctuation in the sealed space. In particular, it is possible to estimate the heart rate even in a state in which body movement occurs.

By extracting, as waveform feature points, zero crossing points having a fluctuation width equal to or greater than a threshold value from a signal of a waveform representing pressure fluctuation in a sealed space, the estimation accuracy of a heart rate is improved.

By using a discrete Fourier transform in frequency analysis of pulse waveforms, a significant reduction in computational complexity is realized compared to cases using a fast Fourier transform.

In the technique of the present disclosure, the waveform feature points are extracted from a waveform representing pressure fluctuation in a sealed space by a feature point extraction unit. A pulse waveform generation unit generates a pulse waveform in which pulses are formed at the same timing as the waveform feature points. A frequency analysis unit performs frequency analysis on the pulse waveform. A heart rate is estimated by a heart rate estimation unit by analyzing cyclic feature points obtained from the results of the frequency analysis.

In this manner, waveform feature points are extracted from waveforms representing pressure fluctuation in a sealed space, a pulse waveform is generated in which pulses are formed at the same timing as the waveform feature points, and a heart rate is estimated by analyzing cyclic feature points obtained from the results of performing frequency analysis on the pulse waveform. This enables the heart rate to be accurately estimated from pressure fluctuation in the sealed space.

The feature point extraction unit according to the technique of the present disclosure can extract, from a waveform representing pressure fluctuation in a sealed space, a zero crossing, a peak point, or a trough point having a fluctuation width equal to or greater than a threshold value, as a waveform feature point.

The pulse waveform generation unit according to the technique of the present disclosure can generate a pulse waveform in which a pulse of a predetermined amplitude is formed.

The frequency analysis unit according to the technique of the present disclosure can perform the frequency analysis using a discrete Fourier transform.

The headset system according to the technique of the present disclosure is configured including the above-described heart rate estimation device, and a headset including a hollow housing worn at an ear of a user, a driver for sound signal output provided inside the housing, a microphone provided inside the housing and rearward of the driver, and an output unit that outputs a signal output from the microphone to the heart rate estimation device as a waveform representing pressure fluctuation in a sealed space.

According to the technique of the present disclosure, in a headset, a signal output from the microphone by an output unit is output to the heart rate estimation device as a waveform representing pressure fluctuation in a sealed space.

Further, the heart rate estimation device estimates the heart rate from the signal output from the microphone.

In this manner, the heart rate can be accurately estimated from pressure fluctuation in the sealed space, using the signal output from the microphone as a waveform representing pressure fluctuation in the sealed space.

According to the technique of the present disclosure, in a headset, a signal output from the optical detection unit by an output unit is output to the heart rate estimation device as a waveform representing pressure fluctuation in a sealed space.

Further, the heart rate estimation device estimates the heart rate from the signal output from the optical detection unit.

In this manner, the heart rate can be accurately estimated from pressure fluctuation in the sealed space, using the signal output from the optical detection unit as a waveform representing pressure fluctuation in the sealed space.

### Second Exemplary Embodiment

Explanation follows regarding a headset system according to a second exemplary embodiment. Portions that have the same configuration as those in the first exemplary embodiment are appended with the same reference numerals, and explanation thereof is omitted.

The second exemplary embodiment differs from the first exemplary embodiment in that biological information is acquired using a photoelectric pulse wave.

### Configuration of Headset System of Second Exemplary Embodiment of Technique of Present Disclosure

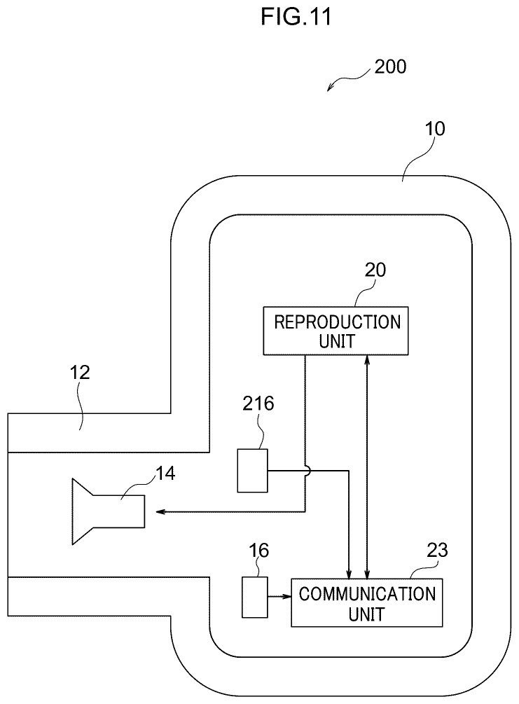

A headset system according to a second exemplary embodiment of the technique of the present disclosure includes the headset 200 illustrated in FIG. 11. The headset 200 has the same configuration as the headset 100 according to the above-described first exemplary embodiment, and further includes a photoelectric pulse wave measuring device 216 that measures a photoelectric pulse wave. The photoelectric pulse wave measuring device 216 is an example of an optical detection unit.

The photoelectric pulse wave measuring device 216 includes a light emitting unit for light such as near-infrared LED and an optical detection unit such as a phototransistor, measures a photoelectric pulse wave inside the external ear canal, and outputs a signal indicating the measured photoelectric pulse wave. The signal output from the photoelectric pulse wave measuring device 216 is transmitted to the information processing unit 50 by the communication unit 23. Here, the signal output from the photoelectric pulse wave measuring device 216 is a waveform representing pressure fluctuation in the sealed space formed between the hollow portion of the housing 10 and the ear of the user, and is, for example, a waveform similar to the waveform illustrated in FIG. 1A described above.

The input unit 60 of the information processing unit 50 receives input of a signal output from the photoelectric pulse wave measuring device 216, that is received from the headset 200.

The feature point extraction unit 72 extracts waveform feature points from the signal output from the photoelectric pulse wave measuring device 216.

Note that the remaining configuration and operation of the headset system according to the second exemplary embodiment are the same as those in the first exemplary embodiment, and explanation thereof is omitted.

As explained above, in the headset system according to the second exemplary embodiment of the technique of the present disclosure, the heart rate estimation device extracts waveform feature points from the signal of a waveform representing pressure fluctuation in a sealed space that is output from the photoelectric pulse wave measuring device of the headset. The heart rate estimation device estimates the heart rate by generating a pulse waveform in which pulses are formed at the same timing as the waveform feature points, and analyzing cyclic feature points obtained from the results of performing frequency analysis on the pulse waveform. This enables the heart rate to be accurately estimated from pressure fluctuation in the sealed space. In particular, it is possible to estimate the heart rate even in a state in which body movement occurs.

### EXAMPLES

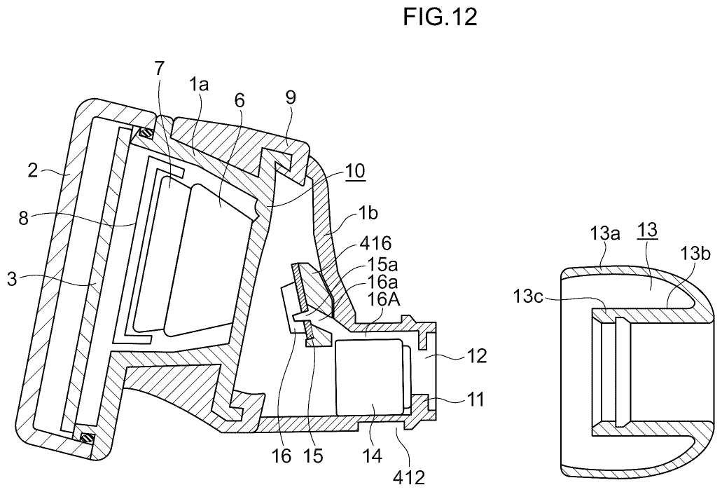

Explanation follows regarding an example of a headset according to the first exemplary embodiment described above. As illustrated in FIG. 12, a housing 10 of the headset of the present exemplary embodiment is formed by fitting together a main housing 1a and a front housing 1b.

The main housing 1a is a hollow member having a cylindrical shape as a whole, in which a rearward opening portion is closed off by a cover 2. A main board 3 is disposed inside the main housing 1a facing the opening portion thereof. The main board 3 is a board on which electronic components functioning as the reproduction unit 20 and the communication unit 23 are mounted.

A battery 6 is disposed in front of the main board 3 with a battery cushion 7 and a battery cap 8 interposed therebetween.

Housing rubber 9 is provided at an outer periphery of the main housing 1a. The housing rubber 9 is a cylindrical resilient member that is fitted into the outer periphery of the main housing 1a, and that alleviates contact with the ear, and prevents water from entering the housing 10.

The front housing 1b is disposed so as to close off a forward opening portion of the main housing 1a that is cylindrical in shape. The front housing 1b has the overall shape of a truncated oblique cone, and a peripheral edge portion thereof is slightly raised toward the side of the tympanic membrane.

An external ear canal insertion portion 12 that projects toward the tympanic membrane side from a top portion of the truncated oblique cone is provided at the front side of the front housing 1b. The external ear canal insertion portion 12 is a cylindrical shape provided at a part of the front housing 1b, is open at the front and rear sides, and communicates the interior of the front housing 1b with the exterior. A driver 14 including a cylindrical case is installed inside the external ear canal insertion portion 12. Thus, a positioning portion 11 of the driver 14 is provided adjacent to the front opening portion of the external ear canal insertion portion 12, and the driver 14 is fixed to the inside face of the external ear canal insertion portion 12 as a result of the front end portion of the driver 14 engaging with the positioning portion 11. A rear end portion of the driver 14 is disposed so as to approach the vicinity of a front end portion of the front housing 1b. The driver 14 includes a magnetic circuit, a diaphragm, and the like for generating an output signal inside a cylindrical case, and any suitable known structure is used.

The headset of the present exemplary embodiment includes a microphone 16. The microphone 16 is provided in the vicinity of the external ear canal insertion portion 12 inside the front housing 1b; namely, at the rear of the driver 14.

The microphone 16 is mounted on a microphone substrate 15. The microphone substrate 15 is fixed to a block 416. The block 416 is a block-shaped member that supports the microphone 16 and the microphone substrate 15. Opening portions 15a, 16a are provided in the microphone substrate 15 and the block 416 so that acoustic signals inside the external ear canal can reach the microphone 16.

A hollow portion 16A, serving as a groove formed along the axial direction of the external ear canal insertion portion 12, is provided at the inside face of the external ear canal insertion portion 12. The hollow portion 16A is a square groove-type space formed between the inside face and a side face of the driver 14, and communicates a front end portion of the external ear canal insertion portion 12 with the opening portion 16a of the block 416 fixed to the front housing 1b.

An earpiece 13 is fixed to the outer circumference of the ear canal insertion portion 12. The earpiece 13 is also referred to as an ear chip, an ear pad, or an ear cap, and is made of a resilient member, such as silicone rubber, for example. The earpiece 13 includes an adhesion portion 13a with respect to an external ear canal wall face, and is formed in a hemisphere at a leading end of a cylindrical portion 13b that is fitted into the outer circumference of the external ear canal insertion portion 12. An earpiece attachment groove 412 is provided at the outer circumference of the external ear canal insertion portion 12, a fitting portion 13c is provided at the inner circumference of the cylindrical portion 13b of the earpiece 13, and the fitting portion 13c is engaged with the earpiece attachment groove 412, such that the earpiece 13 is fixed to the external ear canal insertion portion 12.

As explained in the exemplary embodiments described above, the configuration is such that the microphone is disposed rearward of the driver. As a result, with respect to the volume of the sealed space configured by the housing of the headset accommodating the required configuration components and by the external ear canal, the microphone can be made smaller than in cases in which it is disposed in front of the driver.

Note that with respect to the sealed space formed by the housing of the headset and the external ear canal, the smaller the volume of the sealed space is, the greater becomes the pressure fluctuation including the inflation and contraction of a blood vessel accompanying blood flow (see Reference Document 2). Therefore, pressure fluctuation in the sealed space can be more accurately acquired, and the heart rate can be accurately estimated, as a result of reducing the volume of the sealed space using the configuration explained in the exemplary embodiments described above.

[Reference Document 2]: Japanese Patent No. 6082131

Note that the technology of the present disclosure is not limited to the exemplary embodiments described above, and various modifications and applications are possible within a range that does not depart from the gist of the technique of the present disclosure.

For example, in each of the exemplary embodiments described above, examples of cases in which the technique of the present disclosure is applied to a headset have been explained; however, the present disclosure is not limited thereto. The technique of the present disclosure may have applications other than to a headset, and the technique of the present disclosure may be applied, for example, to an electronic stethoscope.

The disclosure of Japanese Patent Application No. 2022-171796 is hereby incorporated by reference in its entirety.

All documents, patent applications, and technical standards described herein are hereby incorporated by reference to the same extent as if each document, patent application, and technical standard were specifically and individually described as being incorporated by reference.

1. A heart rate estimation device, comprising:
a memory; and
at least one processor coupled to the memory, the at least one processor being configured to:
extract a waveform feature point from a waveform expressing pressure fluctuation in a sealed space;
generate a pulse waveform forming a pulse at a same timing as the waveform feature point;
execute frequency analysis of the pulse waveform; and
estimate a heart rate by analyzing a frequency feature point obtained from a result of the frequency analysis.

2. The heart rate estimation device of claim 1, wherein the at least one processor is configured to extract, as the waveform feature point, a zero crossing having a fluctuation range equal to or greater than a threshold value, a peak point or a trough point from a waveform expressing pressure fluctuation in a sealed space.

3. The heart rate estimation device of claim 1, wherein the at least one processor is configured to generate the pulse waveform forming a pulse of a predetermined amplitude.

4. The heart rate estimation device of claim 1, wherein the at least one processor is configured to execute the frequency analysis by discrete Fourier transform.

5. A headset system, comprising:
the heart rate estimation device of claim 1; and
a headset, the headset including:
a hollow housing to be worn at an ear of a user;
a driver for acoustic signal output which is provided at an interior of the housing;
a microphone provided at the interior of the housing and rearward of the driver; and
an output unit that outputs, to the heart rate estimation device, as a waveform expressing pressure fluctuation in a sealed space, a signal output from the microphone.

6. A headset system, comprising:
the heart rate estimation device of claim 1; and
a headset, the headset including:
a hollow housing to be worn at an ear of a user;
a driver for acoustic signal output which is provided at an interior of the housing;
an optical detection unit provided at the interior of the housing and rearward of the driver; and
an output unit that outputs, to the heart rate estimation device, as a waveform expressing pressure fluctuation in a sealed space, a signal output from the optical detection unit.
